(19) United States
(12) Patent Application Publication
(10) Pub. No.: US 2008/0194525 A1
(43) Pub. Date: Aug. 14, 2008

(54) Process of Making Geminal Bisphosphonic Acids and Pharmaceutically Acceptable Salts and/or Hydrates Thereof

(76) Inventors: Jordi Puig Serrano, Girona (ES); Jordi Bosch Illado, Girona (ES)

Correspondence Address: FROMMER LAWRENCE & HAUG, 745 FIFTH AVENUE- 10TH FL., NEW YORK, NY, 10151, US

(21) Appl. No.: 11/913,695

(22) PCT Filed: May 5, 2006
(86) PCT No.: PCT/IB06/02169

§ 371 Date: Feb. 21, 2008

Related U.S. Application Data
(60) Provisional application No. 60/678,425, filed on May 6, 2005.

(51) Int. Cl. A61K 31/675; C07F 9/28; A61P 3/14

(52) U.S. Cl. 514/89; 562/22

(57) ABSTRACT

This invention relates to the process of making geminal bisphosphonic acids and pharmaceutically acceptable salts and/or hydrates thereof which have the formula (I): wherein X¹, X², X³, X¹R¹ and R² are described herein.

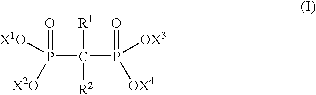

### RELATED APPLICATION(S)/INCORPORATION BY REFERENCE

This application claims priority from U.S. Provisional Application No. 60/678,425, filed May 6, 2005. The foregoing application(s), and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

This invention relates to the process of making geminal bisphosphonic acids and pharmaceutically acceptable salts and/or hydrates thereof which have the formula:

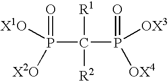

wherein X¹, X², X³, X⁴, R¹ and R² are described herein.

### BACKGROUND OF THE INVENTION

Geminal bisphosphonic acids and pharmaceutically acceptable salts (the “bisphosphonates”) have been studied for use in the treatment of Paget's disease and hypercalcemia but much interest has concerned their role in the treatment of osteoporosis (e.g. alendronate (FOSAMAX®). Other previously known bisphosphonates include clodronate, etidronate, etidronate sodium, ibandronate, pamidronate, risedronate, tiludronate and zoledronic acid.

Risedronic acid and its pharmaceutically acceptable salts are described in U.S. Pat. No. 5,583,122. No examples for the preparation of specific risedronic acid or its pharmaceutically acceptable salts are included. However, a method for obtaining 2-(2-pyridyl)-1-hydroxy-ethane-1,1-diphosphonic acid, an isomer of position of risedronic acid, is described. The method makes use of a mixture of phosphorous acid and phosphorous trichloride, as bisphosphonation mixture, in chlorobenzene. The crude bisphosphonic acid is obtained from a mixture of water and methanol and then is recrystallized from hot water.

Example 3 of U.S. Pat. No. 5,391,743 (equivalent to EP 646 119 B1) refers to a preparation of risedronic acid. According to this example, a mixture of 3-pyridylacetic acid hydrochloride, phosphorous acid and phosphorous trichloride is refluxed at 100° C. in the presence of chlorobenzene and a gummy oil is formed. The chlorobenzene is decanted and the gummy oil is hydrolyzed in 1N HCl. The obtained crystals are then filtered. The filtrate is evaporated to an oil and a small amount of water is added to dissolved the oil. Then ethanol is added to induce crystallization. The two crops of solids obtained are recrystallized from hot water. No characteristics of the obtained product are mentioned.

The paper published in J. Org. Chem. (1995), 60(25), 8310-12 refers to a preparation of risedronic acid monohydrate using methanesulphonic acid as a diluent. The obtained product is characterized by its ¹H-NMR, ¹³C-NMR, elemental analysis and content of water (measured by Karl Fischer method).

EP 1 243 592 A refers to a process for the preparation of risedronic acid by refluxing/heating a mixture of 3-pyridylacetic acid, phosphorous acid and phosphorous trichloride in the presence of a halobenzene (chlorobenzene or fluorobenzene), decanting the halobenzene, refluxing the reaction residue with NaOH, and adding concentrated HCl. The solid is filtered and washed with ethanol and dried at 80° C.

Examples 1 and 2 of U.S. Pat. No. 6,562,974 (equivalent to EP 1 252 169 B1) refer to a process for obtaining risedronic acid. According to these examples, the reaction is performed by heating a mixture of 3-pyridyl acetic acid or its hydrochloride, phosphorous acid and phosphorous trichloride in the presence of a cyclic amine or its hydrochloride salts. The reaction is hydrolyzed by heating it with aqueous hydrochloric acid. The crude risedronic acid is recrystallized from aqueous acid/isopropanol. No characteristics of the obtained product are mentioned.

WO 03/093282 refers to a process for obtaining bisphosphonic acids using a bisphosphonation mixture of acids and phosphorous trichloride characterized in that the reaction is conducted in ionic liquids as the reaction solvents. According to the example 3 of this publication, example 2 refers to a preparation of risedronic acid when using the appropriate starting material.

Examples 1-10 of WO 03/097655 refer to a process for obtaining crystalline risedronic acid monohydrate although any characteristics of the obtained product is mentioned. The process makes use of a bisphosphonation mixture phosphorous acid and a selected halophosphorous compound in the presence of a diluent that is an aromatic. The risedronic acid is obtained from a mixture of water and ethanol. This document refers to a method to avoid solidification of the mixture which includes the use of ortho-phosphoric acid or poly(dimethysiloxane) as a codiluent or, alternatively, the use of diatomite silica as a solid support.

WO 04/067541 refers to a process of obtaining risedronic acid monohydrate using a bisphosphonation mixture of phosphorous acid and phosphorous pentoxide in the presence of methanesulphonic acid. The product is obtained by recrystallization from water.

It is believed that the above references are representative of the state of the art with respect to direct phosphonation of 3-pyridyl acetic acid or its hydrochloride using phosphonation reactants. WO 05/012314 refers to the preparation of risedronic acid sodium salt in its hemipentahydrate form by adjusting the pH of solution of risedronic acid in a mixture of water and isopropanol.

Examples 1 and 2 of U.S. Pat. No. 6,410,520 (equivalent to EP 1 252 170 B1) refers to a first preparation of risedronate monosodium salt in the form of its hemipentahydrate or in the form of its monohydrate by adding a selected solvent over an aqueous solution of risedronic acid sodium. According to the text of the patent, the obtained risedronic sodium salts hemi-pentahydrated and monohydrated have been characterized by X-ray powder diffraction, thermogravimetric analysis, DSC, FT-IR and NIR-IR, however no characteristics are included in this patent.

However, it is believed that heretofore processes for making bisphosphonic acids suffered from drawbacks including formation of a sticky mass precipitation when phosphorus halides are employed; problems with laboratory to industrial scale-up; problems with process safety; and not being environmentally friendly.

### SUMMARY OF THE INVENTION

The present invention endeavors to address problems of prior processes for preparing bisphosphonic acids

Geminal bisphosphonic acids and their pharmaceutically-acceptable salts have been proposed for use in the treatment of diseases of bone and calcium metabolism. In particular bisphosphonates, such as:
the sodium salt of 3-amino-1-hydroxy-propylidene-diphosphonic acid. (PAMIDRONATE is its International Nonproprietary Names (INN)),
the sodium salt of 4-amino-1-hydroxybutylidene-1,1-bisphosphonic acid (ALENDRONATE is its International Nonproprietary Names (INN)) and
the sodium salt of 1-hydroxy-2-(3-pyridinyl)ethylidene-1,1-bisphosphonic acid (RISEDRONATE is its International Nonproprietary Names (INN)) and
the sodium salt of [1-hydroxy-2-(1H-imidazol-1-yl)ethylidene]-bisphosphonic acid (ZOLEDRONATE is its International Nonproprietary Names (INN))
The sodium salt of [1-hydroxy-3-[methyl(pentyl)amino]-1-phosphonopropyl]-bisphosphonic acid (IBANDRONATE is its International Nonproprietary Names (INN))
   have been approved by the FDA for use in the treatment of diseases of bone and calcium metabolism.

It is known in the literature that some bisphosphonic acids and their salts are capable of forming hydrates. For example,
ALENDRONATE exists at least in anhydrous form and trihydrate form.
PAMIDRONATE exists at least in anhydrous form and pentahydrate form.
RISEDRONATE exists at least in anhydrous form, hemi-pentahydrate form and monohydrate form. The monohydrate and hemi-pentahydrate are preferred.
RISEDRONIC acid exists at least in anhydrous form and monohydrate form.
ZOLEDRONIC acid exists at least in anhydrous form and monohydrate.
IBANDRONATE acid exist at least in monohydrate form.

The present invention relates to a novel process for making geminal bisphosphonic acids and their pharmaceutically acceptable salts. The process of the invention provides a novel bisphosphonation method advantageously characterized by using a mixture comprising or consisting essentially of phosphorous acid and a phosphonic anhydride such as propylphosphonic anhydride (also known as 1-propanephosphonic anhydride or 2,4,6-tripropyl-1,3,5,2,4,6-trioxatriphosphorinane-2,4,6-trioxide). The process of the invention also provides a novel bisphosphonation method advantageously characterized by use of an agent comprising or consisting essentially of methanesulfonic anhydride. The process of the invention additionally provides a novel bisphosphonation method advantageously characterized by use of a mixture comprising or consisting essentially of methanesulfonic anhydride and phosphorous acid.

As used herein, “bisphosphonation” is the chemical reaction resulting in the production of a product containing two phosphonic acid groups on the same carbon (geminal bisphosphonic compounds).

In one embodiment of the invention, the geminal bisphosphonic acids made by this process are 1-hydroxy-2-(3-pyridinyl)ethylidine bisphosphonic acid (risedronic acid), 4-amino-1-hydroxybutylidene-1,1-bisphonic acid (aledronic acid), 3-amino-1-hydroxypropylidene-diphosphonic acid (pamidronic acid), [1-hydroxy-2-(1H-imidazol-1-yl)ethylidene]-bisphosphonic acid (zoledronic acid) and [1-hydroxy-3-[methyl(pentyl)amino]-1-phosphonopropyl]-bisphosphonic acid (ibandronic acid). In another embodiment of the invention, the geminal bisphosphonic acid is risedronic acid, 1-hydroxy-2-(3-pyridinyl)ethylidene-1,1-bisphosphonic acid.

The method for making geminal bisphosphonic acids and their pharmaceutically acceptable salts of the invention is advantageous for reasons that include but are not limited to:
(1) The sticky mass that precipitates out when phosphorous halides instead of phosphonic anhydrides are used is avoided.
(2) A robust scalable process is provided. Significant variation on temperature and reaction time do not significantly affect both purity and yield which makes the described process very suitable for industrial scale up.
(3) High yield resulting in no significant loss of starting material.
(4) The process using propylphosphonic anhydride is safer and much more environmentally friendly than the one using phosphorous halides.
(5) The use of a class III solvent such as ethyl acetate implies a safer, greener and environmentally-friendly process.

It is believed that the use of phosphonic anhydrides instead of phosphorous halides or methanesulfonic anhydride has not been reported previously in the literature for this reaction, nor has the use of alkyl-phosphonic anhydride derivatives. And, it is further believed that the use of esters (in particular ethyl acetate) as a solvent in the synthesis of bisphosphonates has not been published until now.

In view of the easier handling, safety benefits and environmental advantages, the use of propylphosphonic anhydride represents a surprising and unexpected improvement when compared with the known methods for the synthesis of bisphosphonates.

For the purposes of this invention, the term “industrial scale-up” defines a process which produces geminal bisphosphonic acid in amounts selected from the ranges of greater than about 100 g, greater than about 1 kg, and greater than about 100 kg; and the term “substantially solution form” defines that the reactants/reaction mixture have a solids content selected from the ranges of less than about 10% w/w, less than about 5% w/w and less than about 1% w/w.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are apparent from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 depicts the X-ray powder diffractogram of risedronic acid obtained in Example D3.

FIG. 2 depicts the X-ray powder diffractogram of risedronate sodium hemipentahydrate obtained in Example 1.

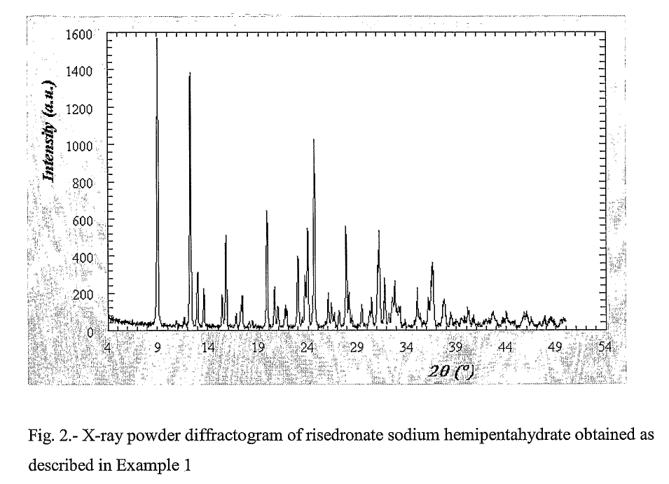

### DETAILED DESCRIPTION

The present invention relates to a novel process for making geminal bisphosphonic acids and their pharmaceutically acceptable salts. The process provides for bisphosphonation using a mixture of phosphorous acid and a phosphonic anhydride such as propylphosphonic anhydride (also known as 1-propanephosphonic anhydride or 2,4,6-tripropyl-1,3,5,2,4,6-trioxatriphosphorinane-2,4,6-trioxide).

Preferred geminal bisphosphonic acid include but are not limited to clodronic acid, etidronic acid, ibandronic acid, pamidronic acid, residronic acid, tiludronic and zoledronic acid.

Particularly preferred geminal bisphosphonic acids made by this process are 1-hydroxy-2-(3-pyridinyl)ethylidine bisphosphonic acid (risedronic acid), 4-amino-1-hydroxybutylidene-1,1-bisphonic acid (aledronic acid), and 3-amino-1-hydroxypropylidene-diphosphonic acid (pamidronic acid), [1-hydroxy-2-(1H-imidazol-1-yl)ethylidene]-bisphosphonic acid (zoledronic acid) and [1-hydroxy-3-[methyl(pentyl)amino]-1-phosphonopropyl]-bisphosphonic acid (ibandronic acid). Most preferred is risedronic acid, 1-hydroxy-2-(3-pyridinyl)ethylidene-1,1-bisphosphonic acid.

The process of the present invention for making geminal bisphosphonic acids and their pharmaceutically acceptable salts involves the phosphonation of a mixture which consists of a compound of the formula R¹—C(═O)—R², phosphorous acid and an alkylphosphonic anhydride and optionally a pH adjustment to form the appropriate salt or hydrate. This process is easily adaptable to industrial application with good yields and does not require the use of reactants such as PCl₃, PCl₅ or POCl₃, which are highly toxic and hazardous for the environment. Furthermore, the use of any of these reagents results in the formation of reaction masses that are difficult to stir and handle.

Pharmaceutically acceptable salts with bases include but are not limited to alkali metal salts (including but not limited to sodium salts or potassium salts), alkaline earth metal salts (including but not limited to calcium salts or magnesium salts) or ammonium salts derived from ammonia or organic amines such as, for example, diethyl amine, triethlyamine, ethyldiisopropylamine, procaine, dibenzylamine, N-methylmorpholine, dihydroabietylamine or methylpiperidine. In one embodiment of the invention, the pharmaceutically acceptable salt form is a sodium or potassium salt.

The presence of an additional inert solvent is optional. Although it is not necessary, the bisphosphonation reaction is advantageously conducted in the presence of inert solvents. An inert solvent is understood to be one which does not react substantially with the reactants involved. Preferred among the inert solvents are an aprotic solvent such as aromatic hydrocarbons (such as xylene, toluene, etc), organic acetates solvents (such as ethyl acetate, isopropyl acetate, etc), ketonic solvents and mixtures thereof. Particularly preferred are toluene and ethyl acetate.

In particular, the invention relates to a process for the preparation of 3-pyridyl-1-hydroxyethylidene-1,1-bisphosphonic acid (risedronic acid) and its pharmaceutically acceptable salts. The process consists of reacting 3-pyridyl acetic acid in its free base form or as a hydrohalide salt with a phosphonation mixture formed by phosphorous acid and propylphosphonic anhydride (also known as 1-propanephosphonic anhydride or 2,4,6-tripropyl-1,3,5,2,4,6-trioxatriphosphorinane-2,4,6-trioxide), and thereafter hydrolyzing the bisphosphonation mixture and the product of said bisphosphonation reaction and isolating directly, by an appropriate pH adjustment, if necessary, the free acid or the sodium salts.

Optionally, once the bisphosphonic acid is obtained, the corresponding pharmaceutically acceptable salt may be obtained by treating a suspension or a solution of the bisphosphonic acid with a base.

Optionally, once the sodium salt has been obtained, the 3-pyridyl-1-hydroxyethylidene-1,1-bisphosphonic acid may be obtained by neutralizing a solution or suspension of the salt with an acid stronger than the 3-pyridyl-1-hydroxyethylidene-1,1-bisphosphonic acid itself, for example, hydrochloric acid, sulfuric acid, etc.

The process for the preparation of 3-pyridyl-1-hydroxyethylidene-1,1-bisphosphonic acid (risedronic acid) and their pharmaceutically acceptable salts, according to the invention, is simple, economic and is readily adapted to industrial production, since it only requires simple industrial operations, the yields obtained are good and the necessary starting raw materials may be acquired on the marketplace.

General Process for Preparing Geminal Bisphosphonic Acid and Salts and/or Hydrates Thereof:

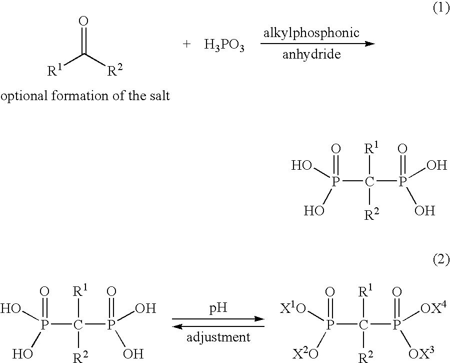

wherein:
X¹, X², X³ and X⁴ are independently a hydrogen or forms a pharmaceutically acceptable salt and

R¹ and R² are independently hydrogen, hydroxy or alkyl which is substituted or unsubstituted. In an another embodiment of the invention, R¹ is hydroxy and R² is a substituted or unsubstituted C₁-C₄-alkyl; in yet another embodiment of the invention, R¹ is hydroxy and R² is a C₁-C₂-alkyl.

In another embodiment of the invention, when the alkyl is substituted, the substitution is at least one moiety selected from the group consisting of amino, aryl or N-containing heteroaryl. In another embodiment of the invention, the heteroaryl is selected from the group consisting of, pyrrolyl, pyrrolinyl, pyrrolidinyl, imidazolyl, imidazolinyl, imidazolidinyl, pyrazolyl, pyrazolinyl, pyrazolidinyl, pyridinyl, piperidinyl, pyridazinyl, pyrimidinyl, pyrazinyl, piperazinyl and triazinyl. In yet another embodiment of the invention, the N-containing heteroaryl is pyridinyl or imidazolyl.

In another embodiment of the invention, the alkylphosphinic anhydride is a C₁-C₈-alkylphosphinic anhydride. In yet another embodiment of the invention, the alkylphosphinic anhydride is a C₁-C₄-alkylphosphinic anhydride. In still another embodiment of the invention, the alkylphosphinic anhydride is propylphosphonic anhydride.

Thus, “alkyl” is generally a straight chain or branched chain unsubstituted or substituted C₁-C₁₂ carbon chain, or a cyclic C₃-C₁₂ substituted or unsubstituted carbon ring, advantageously a straight chain or branched chain unsubstituted or substituted C₁-C₈ carbon chain or an unsubstituted or substituted C₃-C₈ carbon ring, for instance, a straight chain or branched chain unsubstituted or substituted C₁-C₆ carbon chain or an unsubstituted or substituted C₃-C₆ carbon ring, e.g., a straight chain or branched chain unsubstituted or substituted C₁-C₅ or C₁-C₄ carbon chain, such as a straight chain or branched chain unsubstituted or substituted C₁-C₃ or C₁-C₂ carbon chain. Aryl is typically an unsubstituted or substituted C₅ to C₁₂ membered aromatic ring, such as phenyl or tolyl or xylyl. If the alkyl group comprises more than one C then those carbons need not necessarily be linked to each other. For instance, at least two of the carbons of an alkyl may be linked via a suitable element or groupe. g., the alkyl chain or ring or an aromatic ring of an aryl may hetero atoms. Suitable hetero atoms will be apparent to those skilled in the art and include, for instance, sulphur, nitrogen and oxygen. Suitable substitutions on carbon chains or rings, e.g., alkyl carbon chains or rings or aryl rings, include halo, e.g., F, Cl, Br, I, alkoxy, nitro, an alkyl group, a cyclic group such as a cyclo-alkyl or aryl group. Of course, the size of alkyl and aryl groups and substituents therein or thereon should be non-interfering or minimally interfering as to the inventive process.

In yet another embodiment of the invention the process for preparing geminal bisphosphonic acid or hydrated form thereof thereof according to the invention comprises the following steps:
(i) reacting a compound of R¹—C(═O)—R² with a phosphonation mixture formed by phosphorous acid and an alkylphosphonic anhydride, in an inert reaction media. The reactants are added to the reaction vessel.
   In one embodiment of the invention the ratio of moles for phosphorous acid: alkylphosphonic anhydride is about 0.5:1 to about 3:1. In another embodiment of the invention, the ratio is about 1:1 to about 2:1.

Appropriate temperatures for the bisphosphonation reaction is selected from the range of from about 35° C. to about 125° C. and from about 65° C. to about 95° C.

The mixture is stirred at the required temperature for the required time.
(ii) hydrolyzing the reaction mixture with water,

Once the bisphosphonation reaction is complete, it is interrupted by the addition of water at a temperature selected from the range from about 0° C. to about 90° C., preferably from about 0° C. to about 40° C. to hydrolyze unreacted bisfosphonation mixture. In those cases where the addition of water results in the formation of a biphasic system, the organic layer is preferably decanted and separated. In every case, the resulting aqueous mixture is heated to a temperature above about 50° C., preferably to the reflux temperature of said mixture, to ensure complete hydrolysis.
(iii) isolating bisphosphonic acid or hydrated form thereof,
(iv) optionally adjusting the pH to form the pharmaceutically acceptable salt;
(v) optionally, purifying the bisphosphonic acid or hydrated form thereof or pharmaceutically acceptable salt.

### Process for Preparing Risedronic Acid:

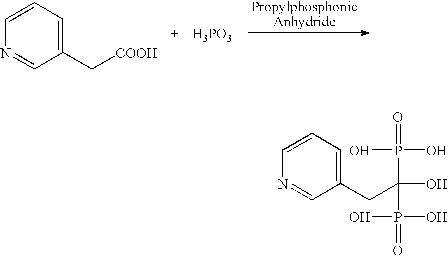

One embodiment of the process for preparing risedronic acid and/or hydrates thereof according to the invention comprises the following steps:
(i) reacting pyridine-3-ylacetic acid hydrochloride with a phosphonation mixture formed by phosphorous acid and propylphosphonic anhydride, (or also 1-propanephosphonic anhydride, or also 2,4,6-tripropyl-1,3,5,2,4,6-trioxatriphosphorinane-2,4,6-trioxide) in an inert reaction media. The reactants are added to the reaction vessel.

In one embodiment of the process, about 3 moles of phosphorous acid are used and about 2.5 moles of propylphosphonic anhydride (6:5 ratio) are used. In another embodiment of the experiments using about 2.5 moles of phosphorous acid and about 1.5 moles of propylphosphonic anhydride (5:3 ratio) also gave an acceptable yield. The reactants may be added to the reaction vessel and the mixture heated up to react (batch mode reaction) or, in another embodiment of the invention the propylphosphonic acid solution is added stepwise into the reactor vessel containing the rest of the reactants and a convenient amount of the convenient solvent (semi-batch mode). The semi-batch mode is more convenient since the thermal response of the reaction may be addition controlled. The semi-batch mode is characterized for the addition time. A convenient addition time is between about several minutes to about several hours. In one embodiment of the addition time, the time is between about 5 to about 15 hours.

Alternatively, the pyridine-3-yl acetic acid hydrochloride can be reacted with methanesulfonic anhydride or methanesulfonic acid and phosphorus acid,
Appropriate temperatures for the bisphosphonation reaction is selected from the ranges of from about 35° C. to about 125° C. and from about 65° C. to about 95° C.
The mixture is stirred at the required temperature for the required time.
(ii) hydrolyzing the reaction mixture with water,
   Once the bisphosphonation reaction is complete, it is interrupted by the addition of water at a temperature range selected from about 0° C. to about 90° C. and from about 0° C. to about 40° C. to hydrolyze unreacted bisphosphonation mixture. In those cases where the addition of water results in the formation of a biphasic system, the organic layer is preferably decanted and separated. In every case, the resulting aqueous mixture is heated to a temperature range selected from above 50° C. and the reflux temperature of said mixture, to ensure complete hydrolysis.
(iii) optionally adjusting the pH to form the pharmaceutically acceptable salt; or, alternatively,
(iv) isolating risedronic acid, and
(v) optionally, purifying the risedronic acid by recrystallizing it from hot water.

In a further embodiment of the invention, the bisphosphonic acids and/or hydrates thereof formed by the described process results in a yield of at least about 70%; in another embodiment of the invention, the yield is in the range of about 70% to about 90.%.

In a further embodiment of the invention, the chromatographic purity of the bisphosphonic acids and/or hydrates thereof formed by the described process is at least 99.00%; in another embodiment of the invention, the chromatographic purity is about 99.25% to about 99.6%

The above described embodiments of yields and chromatographic purity can also be simultaneously present in the bisphosphonic acids formed by the described processes.

The bisphosphonic acids formed by the process described above can further be treated to form the corresponding salt and/or hydrate form thereof.

In one embodiment of the invention, the process for converting bisphosphonic acid or its hydrated forms thereof to its salt thereof comprises:
(i) converting bisphosphonic acid or a hydrate thereof to its salt by treatment with an ion source and adjusting the pH between about 4.5 to about 5.0
(ii) isolating the sodium salt of bisphosphonic acid, and
(iii) optionally, purifying the sodium salt of bisphosphonic acid.

In another embodiment of the invention, the process for converting bisphosphonic acid or its hydrated forms thereof to its salt thereof comprises:
(i) treating the bisphosphonic acid or its hydrated form thereof with an organic amine to form the corresponding ammonium salt;
(ii) converting the ammonium salt to an ion salt by contacting with a sodium ion source, such as a sodium halide and adjusting the pH between about 4.5 to about 5.0;
(iii) isolating the salt of bisphosphonic acid or its hydrated form thereof, and
(iv) optionally, purifying the salt of bisphosphonic acid or its hydrated form thereof.

In one embodiment of the invention the organic amine is an arylamine or a mono-, di- or tri-alkylamine. In another embodiment of the invention the alkyl length of the mono-, di- or tri-alkylamine is C₁-C₈; in yet another embodiment of the invention the alkyl length of the mono-, di- or tri-alkylamine is C₁-C₄.

The advantages of these herein described methods of forming the salt and/or hydrate thereof of a bisphophonic acid or its hydrated form thereof include:
(1) a crystallization rate which is easily controlled by the addition of an aqueous solution of sodium chloride. This results in an easy and cheap way to regulate the hydration degree.
(2) an easier filtration of insoluble particles which can be performed at room temperature in aqueous media.
(3) No heating step is required.

Another embodiment of the invention are pharmaceutical compositions containing the hemipentahydrate or the monohydrate forms of monosodium salt of bisphosphonic acid or mixtures thereof.

In a further embodiment of the invention, the salts of the bisphosphonic acids or hydrated form thereof formed by the described process results in a yield of at least about 70%; in another embodiment of the invention, the yield is in the range of about 75% to about 85%.

In a further embodiment of the invention, the chromatographic purity of the salts of bisphosphonic acids or hydrated form thereof formed by the described process is at least 99.50%; in another embodiment of the invention, the chromatographic purity is about 99.7% to about 99.9%.

The above described embodiments of yields and chromatographic purity can also be simultaneously present in the salts of bisphosphonic acids or hydrated form thereof formed by the described processes.

### Preparation of Risedronic Acid:

The following descriptions correspond to four different approaches to the synthesis of risedronic acid.

The first approach (Approach A, example A1) exemplifies the synthetic procedures adapted from literature that use phosphorous halides. From these descriptions, it should be fairly clear that this process is not suitable to scale up, and accordingly improvement is required. Some advances have recently been published, as described in the table above, providing methodologies that avoid the solidification problem. These methods can of course be scaled up, however a general method that provides safety and environmentally friendly performance is still required.

Procedures B, C and D, as described herein, provide synthetic alternatives that have increased safety and are more environmentally friendly in comparison to the other methodologies currently in use.

The second approach (Approach B, example B1) refers to an adaptation of the process of U.S. Pat. No. 6,573,401 (assigned to Medichem S.A. and which is incorporated by reference), which uses methanesulfonic anhydride instead of the phosphorous halides.

The third approach is the “combined” approach (approach C, examples C1 and C2) and it describes a way of avoiding solidification by using methanesulfonic anhydride together with phosphorous halides. Here the methanesulfonic anhydride plays the double role of dehydrating agent and stirring enhancer.

The fourth approach (approach D, examples D1 to D4) describes the propylphosphonic anhydride approach. The procedure depicted is a cleaner, environmentally friendly methodology that appears to be easy to scale up and which uses materials that are easier to handle than the phosphorous halides. There are no solidification problems during the reaction, yields are high, reaction times affordable and the reaction is simple to work up.

### Process for Preparing Sodium Salts of Risedronic Acid and its Hydrated Forms

According to the present invention, two methods are described:

### Method 1:

(i) converting solid risedronic acid or a hydrate thereof to its sodium salt by treatment with a sodium ion source, such as sodium inorganic or organic bases, most preferably sodium hydroxide, and adjusting the pH between about 4.5 to about 5.0
(ii) isolating the sodium salt of risedronic acid, and
(iii) optionally, purifying the sodium salt of risedronic acid

### Method 2:

Risedronic acid or a hydrate thereof is converted to its sodium salt by first treating the acid with an organic amine, to form the corresponding ammonium salt, and then converting the ammonium salt to the monosodium salt by contacting with a sodium ion source, such as a sodium halide, preferably sodium chloride, and adjusting the pH between about 4.5 to about 5.0.

In another embodiment of method 2, the target risedronate monosodium salt hemipentahydrate is obtained in a procedure comprising:
(1) The reactants are added to the reaction vessel:
   Risedronic acid, water and about 1.5 equivalent/mol of an organic amine (such as alkylamines or arylamines, preferably triethylamine or diethylamine) are charged into the reaction vessel. The mixture is stirred at about 20-30° C. for about 15-30 minutes to solubilize or almost solubilize.
(2) Filtration.

The solution or resulting mixture can be optionally treated with a decolorating agent and is filtered in order to separate insoluble particles.
(3) The filtrate is collected and pH is adjusted between about 4.5 to about 5.0, most preferably to about 4.7.
(4) The mixture is stirred for about 1 h in order to equilibrate.
(5) pH is readjusted to about 4.7 (optional, only when necessary).
(6) Precipitating solvent is added to the aqueous solution, most preferably acetone or isopropyl alcohol.
(7) An aqueous salt solution, e.g., a solution of NaCl, is poured into the solution.
(8) The mixture is stirred, and the precipitated solid is collected by filtration.
(9) Optionally, the salt, e.g., sodium salt, of risedronic acid is purified.

To the best of the knowledge of Applicant(s), no process using any organic amine has been published, and also no process that filters insoluble particles in a 100% aqueous solution. Furthermore, in all examples described in the literature, heating is needed to obtain the desired hydration degree for the risedronate salt (e.g., sodium salt).

Another embodiment of the invention are pharmaceutical compositions containing the hemipentahydrate or the monohydrate forms of monosodium salt of risedronic acid or mixtures thereof.

The invention will now be further described by way of the following non-limiting examples. The following examples are given for the purpose of providing those skilled in the art with a sufficiently clear and complete explanation of the present invention, but must not be considered as limitations on the essential aspects of the object thereof, as set forth in the foregoing paragraphs of this description. In all cases chromatographic data refers to the HPLC methods which follow the examples.

### EXAMPLES

### Approach A. The Phosphorous Halides Approach

### Example A1

### Synthesis of 3-pyridyl-1-hydroxyethylidene-1,1-bisphosphonic Acid

This example is a reproduction of Example 1 in WO 03/097655 A1 and exemplifies the need of a suitable process to scale up.

In a 100 ml rounded bottom, three necked flask equipped with a magnetic stirrer, a thermometer, a reflux condenser and a compensated-pressure dropping funnel were charged: 2 g (1.15·10⁻² mol) of 3-pyridyl acetic acid hydrochloride, 2.92 g (3.56·10⁻² mol) of phosphonic acid and 14.4 ml of toluene.

The mixture was heated to 87° C. at which point 3.23 ml (3.53·10⁻² mol) of POCl₃ was added dropwise during about 20 minutes. The resulting mixture was heated up to 95° C. After a few minutes at regime a sticky solid appeared, disenabling a proper stirring. The mixture was kept at 95° C. 20.5 h in these deficient stirring conditions.

After the regime, the resulting mixture was cooled down to about 65° C. and 14.4 ml of water were added dropwise. Exothermy was observed. The sticky solid was dissolved and the efficient stirring recovered. The mixture was then let to cool down to room temperature and the aqueous layer decanted. These additional steps required to dissolve the sticky solid makes this approach unsuitable for industrial scale up as the reactants are not maintained in substantially solution form prior to the precipitation step.

The aqueous phase was heated up to 90-95° C. and stirred at this temperature overnight. Then, was let to cool down to room temperature and 28.8 ml of ethanol were added and the precipitation of a whitish solid observed. The mixture was cooled down to 5° C. and filtered washing the cake with ethanol. The white solid obtained was dried at 50° C. in a vacuum oven to obtain 2.80 g (9.30·10⁻³ mol) of 3-pyridyl-1-hydroxyethylidene-1,1-bisphosphonic acid monohydrate (Yield: 80.71%) Chromatographic purity: 98.496% (area percent)

### Approach B. The Methanesulfonic Anhydride Approach

### Example B1

### Synthesis of 3-pyridyl-1-hydroxyethylidene-1,1-bisphosphonic Acid

In a 100 ml rounded bottom, three necked flask equipped with a magnetic stirrer, a thermometer, a reflux condenser and a compensated-pressure dropping funnel were charged: 2 g (1.15·10⁻² mol) of 3-pyridyl acetic acid hydrochloride, 2.92 g (3.56·10⁻² mol) of phosphonic acid and 14.4 ml of toluene.

The mixture was heated to 87° C. at which point 6.02 g (3.46·10⁻² mol) of methansulfonic anhydride was steeply added in about 20 minutes. The resulting mixture was heated up to 95° C. and stirred at this temperature for 20.5 h without observing stirring problems.

Then, it was let to cool down to 65° C., at which point 14.4 ml of water were added dropwise in about 20 minutes. The mixture was then let to cool down to room temperature and the aqueous layer separated and heated up to 95° C., stirred overnight at this temperature and let to cool down to room temperature. 28.8 ml of ethanol were then added, the mixture cooled down to 5° C. for 1.2 h and the whitish solid filtered, washing the cake with ethanol.

The solid was dried at 50° C. in a vacuum oven to obtain 1.17 g of 3-pyridyl-1-hydroxyethylidene-1,1-bisphosphonic acid monohydrate (Yield: 33.73%) Chromatographic purity: 95.93% (area percent)

### Approach C. The Combined Phosphorous Halides and Methanesulphonic Anhydride Approach

### Example C1

### Synthesis of 3-pyridyl-1-hydroxyethylidene-1,1-bisphosphonic Acid

In a 100 ml rounded bottom, three necked flask equipped with a magnetic stirrer, a thermometer, a reflux condenser and a compensated-pressure dropping funnel were charged: 1 g (5.76·10⁻³ mol) of 3-pyridyl acetic acid hydrochloride, 1.46 g (1.78·10⁻² mol) of phosphonic acid, 2.01 g (1.15·10⁻² mol) of methanesulfonic anhydride and 7.2 ml of toluene.

The mixture was heated up to 90° C. and 0.54 ml of POCl₃ were added dropwise for about 20 minutes. The mixture was heated up to 95° C. and stirred overnight at this temperature. No stirring problems were observed.

The reaction mixture was let to cool down to 80° C. and 14.4 ml of water were added dropwise. The resulting mixture was stirred at 80° C. for 1 h, let to cool down and the aqueous layer decanted and heated to 95° C. for 5.5 h. After that regime, the aqueous layer was let to cool down to room temperature and 14.4 ml of ethanol were added. The resulting mixture was cooled down to 5° C. for 1 h and the solid filtered and washed with ethanol and dried at 40° C. in a vacuum oven until constant weight. 1.07 g of 3-pyridyl-1-hydroxyethylidene-1,1-bisphosphonic acid monohydrate were obtained (Yield: 61.69%) Chromatographic purity: 99.20% (area percent)

### Example C2

### Synthesis of 3-pyridyl-1-hydroxyethylidene-1,1-bisphosphonic Acid

In a 100 ml rounded bottom, three necked flask equipped with a magnetic stirrer, a thermometer, a reflux condenser and a compensated-pressure dropping funnel were charged: 1.00 g (5.76·10⁻³ mol) of 3-pyridyl acetic acid hydrochloride, 1.46 g (1.78·10⁻² mol) of phosphonic acid, 3.01 g (1.72·10⁻² mol) of methanesulfonic anhydride and 7.2 ml of toluene. 1.22 g of PCl₅ was steeply added at room temperature. Exothermy, gas evolution and foaming was observed. The mixture was then heated up to 95° C. At about 40° C., formation of two well defined liquid phases were observed. The mixture was kept overnight at 95° C. No stirring problems arose.

The mixture was then let to cool down to 80° C. at which point 7.2 ml of water were added dropwise (exothermy was observed). The mixture was stirred at 80° C. for 15 minutes and then let to cool down to room temperature.

The aqueous layer was decanted and heated to 95° C. for 5.5 h and let to cool down to room temperature, at which point 7.2 ml of ethanol were added. The resulting mixture was cooled down to 5° C., stirred for 1 h and filtered. The solid washed with ethanol and dried at 40 ° C. in a vacuum oven until constant weight. 1.01 g of 3-pyridyl-1-hydroxyethylidene-1,1-bisphosphonic acid monohydrate are obtained (Yield: 58.23%) Chromatographic purity: 98.327% (area percent)

### Approach D. The 1-Propanephosphonic Anhydride Approach

### Example D1

### Synthesis of 3-pyridyl-1-hydroxyethylidene-1,1-bisphosphonic Acid

In a 100 ml rounded bottom, three necked flask equipped with a magnetic stirrer, a thermometer, a reflux condenser and a compensated-pressure dropping funnel were charged: 1.00 g (5.76·10⁻³ mol) of 3-pyridyl acetic acid hydrochloride, 1,46 g (1.78·10⁻² mol) of phosphonic acid, 4.58 g (1.44·10⁻² mol) of propylphosphonic anhydride and 7.2 ml of toluene.

The heterogeneous mixture was heated up to 95° C. The mixture was stirred at 95° C. overnight and let to cool down to 80° C. at which point 7.2 ml of water were added dropwise. The resulting mixture was stirred at 80° C. for 15 minutes and let to cool down to room temperature.

The aqueous phase was decanted and heated to 90-95° C. for 5.5 h. A white solid precipitated out from during this heating. The resulting mixture was let to cool down to room temperature, cooled down to 5° C. for 1 hour, filtered and the cake washed with water. The white solid obtained was dried into a vacuum oven at 50° C. until constant weight. 1.56 g of 3-pyridyl-1-hydroxyethylidene-1,1-bisphosphonic acid monohydrate were obtained (Yield: 89.94%) Chromatographic purity: 99.63% (area percent)

### Example D2

### Synthesis of 3-pyridyl-1-hydroxyethylidene-1,1-bisphosphonic Acid

In a 100 ml rounded bottom, three necked flask equipped with a magnetic stirrer, a thermometer, a reflux condenser and a compensated-pressure dropping funnel were charged: 1.00 g (5.76·10⁻³ mol) of 3-pyridyl acetic acid hydrochloride, 1.46 g (1.78·10⁻² mol) of phosphonic acid, 4.58 g (1.44·10⁻² mol) of propylphosphonic anhydride and 7.2 ml of toluene.

The heterogeneous mixture was heated up to 75° C. The mixture was stirred at 95° C. overnight and let to cool down to 80° C. at which point 7.2 ml of water were added dropwise. The resulting mixture was stirred at 75° C. for 15 minutes and let to cool down to room temperature.

The aqueous phase was decanted and heated to 90-95° C. for 5.5 h. A white solid precipitated out from the mixture.

The resulting mixture was cooled down to room temperature again and 7.2 ml of ethanol are added. The mixture was further cooled down to 5° C. for 1 hour, filtered and the cake washed with ethanol. The white solid obtained was dried into a vacuum oven at 50° C. until constant weight. 1.30 g of 3-pyridyl-1-hydroxyethylidene-1,1-bisphosphonic acid monohydrate are obtained (Yield: 74.95%) Chromatographic purity: 99.59% (area percent)

### Example D3

### Synthesis of 3-pyridyl-1-hydroxyethylidene-1,1-bisphosphonic Acid

In a 100 ml rounded bottom, three necked flask equipped with a magnetic stirrer, a thermometer, a reflux condenser and a compensated-pressure dropping funnel were charged: 1.00 g (5.76·10⁻³ mol) g of 3-pyridyl acetic acid hydrochloride, 1.46 g (1.78·10⁻² mol) of phosphonic acid, 4.58 g (1.44·10⁻² mol) of propylphosphonic anhydride and 7.2 ml of ethyl acetate. The mixture was heated up to reflux (76° C.) and stirred overnight at this temperature. 7.2 ml of water were added dropwise and the mixture maintained at reflux 86-89° C. for about 5.5 h. During the hydrolysis a white solid precipitated out.

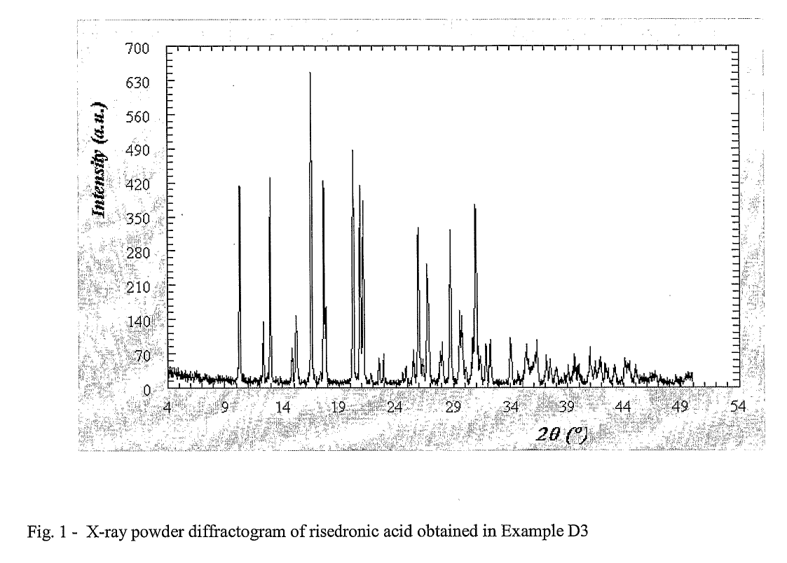

The mixture was let to cool down to room temperature, further cooled down to about 5° C. by means of a water/ice bath and filtered. The white solid obtained was dried into a vacuum oven at 50° C. until constant weight. 1.33 g of 3-pyridyl-1-hydroxyethylidene-1,1-bisphosphonic acid were obtained (Yield: 76.68%) Chromatographic purity: 99.32% (area percent) X-Ray Powder Diffractogram: see FIG. 1

### Example D4

### Synthesis of 3-pyridyl-1-hydroxyethylidene-1,1-bisphosphonic Acid (Semi-Batch Mode Description)

In a 1 liter rounded bottom, three necked flask equipped with a mechanical anchor impeller, a thermometer, a reflux condenser and a compensated-pressure dropping funnel were charged: 40.00 g (2.30·10⁻¹ mol) g of 3-pyridyl acetic acid hydrochloride, 58.68 g (6.91·10⁻¹ mol) of phosphonic acid, and 80 ml of ethyl acetate. The mixture was heated up to reflux (76° C.) at which point addition of 366.4 g of a mixture of propylphosphonic anhydride and ethyl acetate (50% w/w) was added. The addition time was about 10 hours. The mixture was stirred at reflux for 10 additional hours, at which point 200 ml of water was added in about 2 hours. The mixture was hydrolyzed under reflux for about 3.5 hours, let cooled down to 5° C. and the whitish solid precipitated was filtered, the cake washed with water.

After drying at 40° C. in a vacuum oven, 54.26 g of 3-pyridyl-1-hydroxyethylidene-1,1-bisphosphonic acid monohydrate were obtained (Yield: 78.21%) Chromatographic purity: 99.397% (area percent)

### Preparation of Salts of Risedronic Acid

### Example 1

### Synthesis of 3-pyridyl-1-hydroxyethylidene-1,1-bisphosphonate Sodium Hemipentahydrate

In a 250 ml cylindrical flask equipped with a magnetic stirrer, a temperature selector device, and a reflux condenser were charged: 1.50 g (4.98·10⁻³ mol) of (1-hydroxy-2-pyridin-3-ylethane-1,1-diyl)bis(phosphonic acid)monohydrate, 16 ml of water and 0.58 g (7.90·10⁻³ mol) of diethylamine.

The mixture was stirred at room temperature until complete dissolution, pH was adjusted to about 4.7 using HCl 6N and 4.5 ml of isopropyl alcohol were poured into. Then, 0.34 g (5.86·10⁻³ mol) of NaCl dissolved in 2 ml of water was poured into the reaction flask. Precipitation was observed within few seconds.

The solid was collected by filtration and dried in a vacuum oven at 40° C. for 24 h to obtain 1.51 g of risedronate monosodium hemipentahydrate. (84.51% yield) Chromatographic purity: 99.95% (percent area) Water content: 14.60% (KF method) XRD spectra is provided in Figure II. The RXD pattern of the commercially available fully agrees with this pattern disregarding the excipients profile.

### Example 2

### Synthesis of 3-pyridyl-1-hydroxyethylidene-1,1-bisphosphonate Sodium Hemipentahydrate

In a 250 ml cylindrical flask equipped with a magnetic stirrer, a temperature selector device, and a reflux condenser were charged: 1.50 g of (1-hydroxy-2-pyridin-3-ylethane-1,1-diyl)bis(phosphonic acid)monohydrate, 16 ml of water and 0.58 g (7.90·10−3 mol) of diethylamine.

The mixture was stirred at room temperature until complete dissolution, pH was adjusted to about 4.7 using HCl 6N and 4.5 ml of acetone were poured into. Then, 0.34 g (5.86·10−3 mol) of NaCl dissolved in 2 ml of water was poured into the reaction flask. Precipitation was observed within few seconds.

The solid was collected by filtration and dried in a vacuum oven at 40° C. for 24 h to obtain 1.40 g of risedronate monosodium hemipentahydrate. (79.44% yield) Chromatographic purity: 99.97% (percent area) Water content: 13.73% (KF method)

### Chromatographic Purity HPLC Method

The chromatographic separation is carried out with an Atlantis dC18, 3 μm. 5 cm×3.0 mm. I.D column.

The mobile phase is prepared by mixing 130 ml of acetonitrile with 870 ml of pH=6.25 buffer, which is prepared from 11 mM NaH₂PO₄ and 1.1 mM of sodium etidronate dissolved in water, adjusting the pH to 6.25 with NaOH. This mobile phase is mixed and filtered through 0.22 μm nylon filter under vacuum.

The chromatograph is equipped with a 263 nm detector and the flow rate is 1.0 ml per minute at room temperature. Inject 20 μl of the test samples prepared dissolving the appropriate amount of sample to obtain 0.5 mg per ml of water for Risedronate samples or 0.5 mg per ml of 0.24% NaOH in water for Risedronic acid samples.

### HPLC Assay Method

The chromatographic separation is carried out with an Atlantis dC18, 3 μm. 5 cm×3.0 mm. I.D column.

The mobile phase is prepared by mixing 130 ml of acetonitrile with 870 ml of pH=6.25 buffer, which is prepared from 11 mM NaH₂PO₄ and 1.1 mM of sodium etidronate dissolved in water, adjusting the pH to 6.25 with NaOH. This mobile phase is mixed and filtered through 0.22 μm nylon filter under vacuum.

The chromatograph is equipped with a 263 nm detector and the flow rate is 1.0 ml per minute at room temperature. Inject 20 μl of the test samples and Reference standard prepared dissolving the appropriate amount of sample to obtain 0.05 mg per ml of water, for Risedronate samples, or 0.05 mg per ml of 0.24% NaOH in water for risedronic acid samples. Calculate the amount of risedronic or risedronate by comparison of the normalized areas.

Having thus described in detail various embodiments of the present invention, it is to be understood that the invention defined by the above paragraphs is not to be limited to particular details set forth in the above description as many apparent variations thereof are possible without departing from the spirit or scope of the present invention.

1. A process of preparing a geminal bisphosphonic acid or salt or hydrate thereof which comprises:
(a) reacting a compound of the formula R¹—C(═O)—R² in the presence of:
(i) phosphorous acid in the presence of an alkylphosphonic anhydride; or
(ii) methanesulfonic anhydride; or
(iii) methanesulfonic acid and phosphorus acid, to form the bisphosphonic acid of the formula: 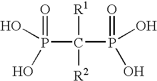
wherein:
R¹ and R² are independently hydrogen, hydroxy or alkyl which is substituted or unsubstituted
(b) optionally adjusting the pH in (a) to form the salt or hydrate of the bisphosphonic acid of the formula: 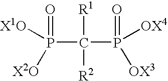
wherein:
R¹ and R² are independently hydrogen, hydroxy or alkyl which is substituted or unsubstituted; and
X¹, X², X³ and X⁴ are independently a hydrogen or forms a pharmaceutically acceptable salt;
(c) optionally, purifying the bisphosphonic acid or salt or hydrate thereof.

2. The process of claim 1, wherein R¹ is hydroxy and R² is a substituted or unsubstituted C₁-C₄-alkyl.

3. The process of claim 2, wherein the moiety when R² is substituted is an N-containing heteroaryl, then the moiety is pyridinyl or imidazolyl.

4. The process of claim 1, wherein the alkylphosphonic anhydride is a C₁-C₈-alkylphosphonic anhydride.

5. The process of claim 4, wherein the alkylphosphonic anhydride is propylphosphonic anhydride.

6. The process of claim 1, wherein the ratio of moles of phosphorus acid:alkylphosphonic anhydride is from about 0.5:1 to about 3:1.

7. The process of claim 6, wherein the ratio of moles of phosphorus acid:alkylphosphonic anhydride is from about 1:1 to about 2:1.

8. The process of claim 1, wherein reacting a compound of the formula R¹—C(═O)—R² is in the presence of phosphorous acid in the presence of an alkylphosphonic anhydride and the chromatographic purity of the bisphosphonic acid formed is at least 99.00%.

9. The process of claim 8, wherein the chromatographic purity is about 99.25% to about 99.6%.

10. The process of claim 9, wherein the yield is in the range of about 75% to about 90%.

11. A process for converting bisphosphonic acid or its hydrated forms thereof to its salt thereof comprises:
(i) converting bisphosphonic acid or a hydrate thereof to its salt by treatment with a sodium ion source and adjusting the pH between about 4.5 to about 5.0
(ii) isolating the sodium salt of bisphosphonic acid, and
(iii) optionally, purifying the sodium salt of bisphosphonic acid; OR
(i) treating the bisphosphonic acid or its hydrated form thereof with an organic amine to form the corresponding ammonium salt;
(ii) converting the ammonium salt to a sodium ion salt by contacting with a sodium ion source, such as a sodium halide and adjusting the pH between about 4.5 to about 5.0;
(iii) isolating the sodium salt of bisphosphonic acid or its hydrated form thereof, and
(iv) optionally, purifying the sodium salt of bisphosphonic acid or its hydrated form thereof.

12. The process of claim 11, wherein the biphosphonic acid or a hydrate thereof is risedronic acid or a hydrate thereof.

13. The process of claim 11, wherein the adjustment of the pH is to about 4.7.

14. The process of claim 11, wherein the organic amine is arylamine or a mono-, di- or tri-alkylamine.

15. The process of claim 11, wherein the ion is a sodium ion.

16. The process of claim 12, wherein the salt formed is a hemi-pentahydrate or monohydrate forms of monosodium, salt of risedronic acid or mixtures thereof.

17. The process of claim 11, wherein the chromatographic purity of the sodium salt of the bisphosphonic acid formed is at least 99.50% and the yield is at least 75%.

18. A hemi-pentahydrate or monohydrate forms of monosodium salt of risedronic acid or mixtures thereof formed by the process of claim 16.

19. A pharmaceutical composition for the treatment of osteoporosis which comprises of the hemi-pentahydrate or monohydrate forms of monosodium salt of risedronic acid or mixtures thereof of claim 18 and a pharmaceutically acceptable carrier.

20. Use of the salt of risedronic acid of claim 18 in the manufacture of a pharmaceutical composition for the treatment of osteoporosis to a patient in need thereof.

21. The process of claim 1 wherein R¹ is hydroxy and R² is 3-pyridinylmethyl.|[MSOFFICE1 ]

22. The process of claim 1 wherein the bisphosphonic acid formed is Risedronic acid.
